(19) United States
(12) Patent Application Publication
(10) Pub. No.: US 2014/0057794 A1
(43) Pub. Date: Feb. 27, 2014

(54) LUNG CANCER SIGNATURE

(71) Applicant: The Regents of the University of Michigan, Ann Arbo, MI (US)

(72) Inventors: David Beer, Chelsea, MI (US); Jeremy Taylor, Ann Arbor, MI (US); Guoan Chen, Ann Arbor, MI (US); Sinae Kim, Fanwood, NJ (US)

(73) Assignee: The Regents of the University of Michigan, Ann Arbor, MI (US)

(21) Appl. No.: 13/972,585

(22) Filed: Aug. 21, 2013

Related U.S. Application Data
(62) Division of application No. 13/473,059, filed on May 16, 2012, now abandoned.
(60) Provisional application No. 61/486,712, filed on May 16, 2011.

(51) Int. Cl. C12Q 1/68

(52) U.S. Cl. CPC C12Q 1/6886
USPC 506/2; 506/16; 506/9; 506/18

(57) ABSTRACT

The present disclosure relates to compositions and methods for cancer diagnosis, research and therapy, including but not limited to, cancer markers. In particular, the present disclosure relates to cancer markers as diagnostic markers and clinical targets for lung cancer.

This application is a divisional of U.S. application Ser. No. 13/473,059, filed May 16, 2012, which claims priority to U.S. Provisional Application No. 61/486,712, filed May 16, 2011, each of which are herein incorporated by reference in its entirety.

### FIELD OF THE INVENTION

The present disclosure relates to compositions and methods for cancer diagnosis, research and therapy, including but not limited to, cancer markers. In particular, the present disclosure relates to cancer markers as diagnostic markers and clinical targets for lung cancer.

### BACKGROUND OF THE INVENTION

Lung cancer remains the leading cause of cancer death in industrialized countries. About 75 percent of lung cancer cases are categorized as non-small cell lung cancer (e.g., adenocarcinomas), and the other 25 percent are small cell lung cancer. Lung cancers are characterized in to several stages, based on the spread of the disease. In stage I cancer, the tumor is only in the lung and surrounded by normal tissue. In stage II cancer, cancer has spread to nearby lymph nodes. In stage III, cancer has spread to the chest wall or diaphragm near the lung, or to the lymph nodes in the mediastinum (the area that separates the two lungs), or to the lymph nodes on the other side of the chest or in the neck. This stage is divided into IIIA, which can usually be operated on, and stage IIIB, which usually cannot withstand surgery. In stage IV, the cancer has spread to other parts of the body.

Most patients with non-small cell lung cancer (NSCLC) present with advanced stage disease, and despite recent advances in multi-modality therapy, the overall ten-year survival rate remains dismal at 8-10% (Fry et al., Cancer 86:1867 [1999]). However, a significant minority of patients, approximately 25-30%, with NSCLC have pathological stage I disease and are usually treated with surgery alone. While it is known that 35-50% of patients with stage I disease will relapse within five years (Williams et al., Thorac. Cardiovasc. Surg. 82:70 [1981]; Pairolero et al., Ann, Thorac. Surg.

38:331 [1984]), it is not currently possible to identify which specific patients are at high risk of relapse.

Adenocarcinoma is currently the predominant histologic subtype of NSCLC (Fry et al., supra; Kaisermann et al., Brazil Oncol. Rep. 8:189 [2001]; Roggli et al., Hum. Pathol. 16:569 [1985]). While histopathological assessment of primary lung carcinomas can roughly stratify patients, there is still an urgent need to identify those patients who are at high risk for recurrent or metastatic disease by other means. Previous studies have identified a number of preoperative variables that impact survival of patients with NSCLC (Gail et al., Cancer 54:1802 1984]; Takise et al., Cancer 61:2083 [1988]; Ichinose et al., J. Thorac. Cardiovasc. Surg. 106:90 [1993]; Harpole et al., Cancer Res. 55:1995]). Tumor size, vascular invasion, poor differentiation, high tumor proliferate index, and several genetic alterations, including K-ras (Rodenhuis et al., N. Engl. J. Med.

317:929 [1987]; Slebos et al., N. Engl. J. Med. 323:561 [1990]) and p53 (Harpole et al., supra; Horio et al., Cancer Res. 53:1 [1993]) mutation, have been reported as prognostic indicators. Tumor stage is an important predictor of patient survival, however, much variability in outcome is not accounted for by stage alone, as is observed for stage I lung adenocarcinoma which has a 65-70% five-year survival (Williams et al., supra; Pairolero et al., supra). Current therapy for patients with stage I disease usually consists of surgical resection and no additional treatment (Williams et al., supra; Pairolero et al., supra). The identification of a high-risk group among patients with stage I disease would lead to consideration of additional therapeutic intervention for this group, as well as leading to improved survival of these patients.

There is a need for additional diagnostic and treatment options, particularly treatments customized to a patient's tumor.

### SUMMARY OF THE INVENTION

The present disclosure relates to compositions and methods for cancer diagnosis, research and therapy, including but not limited to, cancer markers. In particular, the present disclosure relates to cancer markers and panels of cancer markers as diagnostic markers and clinical targets for lung cancer. In some embodiments, the present invention provides compositions, kits, sytems and methods for determinging the likelihood of survival of a subject based on altered expression of one or more cancer markers.

For example, in some embodiments, the present invention provides a kit for characterizing cancer (e.g., determining likelihood of survival) in a subject diagnosed with lung cancer, comprising: reagents for detection of altered expression of one or more (e.g., 2 or more, 3 or more, 4 or more, 5 or more, 10 or more, 25 or more or 50 or more or all of) of A kinase (PRKA) anchor protein 5 (AKAP12), cytochrome P450, family 24, subfamily A, polypeptide 1 (CYP24A1), dual specificity phosphatase 6 (DUSP6), v-erb-b2 erythroblastic leukemia viral oncogene homolog 3 (ERBB3), glyceraldehyde-3-phosphate dehydrogenase (GAPDH), H2A histone family, member Z (H2AFZ), interleukin 11 receptor, alpha (IL11RA), myocyte enhancer factor 2C (MEF2C), O-linked N-acetylglucosamine (GlcNAc) transferase (OGT), ribonucleotide reductase M2 (RRM2), solute carrier family 2 (facilitated glucose transporter), member 1 (SLC2A1), 4-aminobutyrate aminotransferase (ABAT), acetylcholinesterase (ACHE), acyl-CoA synthetase medium-chain family member 3 (ACSM3), adrenergic, beta-2-, receptor, surface (ADRB2), ctivated leukocyte cell adhesion molecule (ALCAM), aryl hydrocarbon receptor nuclear translocator-like 2 (ARNTL2), aurora kinase B (AURKB), basal cell adhesion molecule (Lutheran blood group) (BCAM), baculoviral IAP repeat containing 5 (BIRC5), budding uninhibited by benzimidazoles 1 homolog (yeast) (BUB1), benzodiazapine receptor (peripheral) associated protein 1(BZRAP1), chromosome 1 open reading frame 116 (Clorf116), cyclin B1 (CCNB 1), cyclin-dependent kinase inhibitor 3 (CDKN3), chloride intracellular channel 2 (CLIC2), carbamoyl-phosphate synthase 1, mitochondrial (CPS1), cathepsin L2 (CTSL2), ytoplasmic FMR1 interacting protein 2 (CYFIP2), DEP domain containing 1 (DEPDC1), DNA-damage regulated autophagy modulator 1 (DRAM1), dual specificity phosphatase 4 (DUSP4), epithelial cell transforming sequence 2 oncogene (ECT2), eukaryotic translation initiation factor 4A3 (EIF4A3), epoxide hydrolase 1, microsomal (xenobiotic) (EPHX1), estrogen receptor 1 (ESR1), ets variant gene 5 (ETV5), family with sequence similarity 114, member A2 (FAM114A2), family with sequence similarity 125, member B (FAM125B), Fc fragment of IgG, receptor, transporter, alpha (FCGRT), flap structure-specific endonuclease 1 (FEN1), flavin containing monooxygenase 2 (non-functional) (FMO2), GINS complex subunit 1 (Psf1 homolog) (GINS1), gap junction protein, beta 3, 31 kDa (GJB3), glutaminase (GLS), guanine nucleotide binding protein (G protein), gamma 7 (GNG7), glypican 4 (GPC4), glycerol-3-phosphate dehydrogenase 1-like (GPD1L), G protein-coupled receptor 116 (GPR116), hexokinase 2 (HK2), high mobility group AT-hook 1 (HMGA1), HOP homeobox (HOPX), homeobox D1 (HOXD1), hydroxysteroid (17-beta) dehydrogenase 6 homolog (mouse) (HSD17B6), interleukin 1 receptor, type II (IL1R2), interleukin 6 receptor (IL6R), interaction protein for cytohesin exchange factors 1 (IPCEF1), Kallmann syndrome 1 sequence (KAL1), lipase maturation factor 1 (LMF1), LY6/PLAUR domain containing 3 (LYPD3), mitogen-activated protein kinase 8 interacting protein 3 (MAPK81P3), antigen identified by monoclonal antibody Ki-67 (MKI67), non-SMC condensin II complex, subunit G2 (NCAPG2), non-SMC condensin I complex, subunit H(NCAPH), NLR family, pyrin domain containing 1 (NLRP1), nucleoporin 37 kDa (NUP37), osteomodulin (OMD), parvin, alpha (PARVA), proliferating cell nuclear antigen (PCNA), post-GPI attachment to proteins 3 (PGAP3), plakophilin 2 (PKP2), member RAS oncogene family (RAB36), member RAS oncogene family (RAN), ribosomal protein S6 kinase, 90 kDa, polypeptide 3 (RPS6KA3), RUFY3 (RUFY3), sodium channel, nonvoltage-gated 1, beta (SCNN1B), solute carrier family 34 (sodium phosphate), member 2 (SLC34A2), solute carrier family 47, member 1 (SLC47A1), ST3 beta-galactoside alpha-2,3-sialyltransferase 4 (ST3GAL4), stanniocalcin 2 (STC2), t-complex 1 (TCP1), trefoil factor 1 (TFF1), translocation associated membrane protein 1 (TRAM1), tripartite motif containing 2 (TRIM2), TSC22 domain family, member 3 (TSC22D3), thioredoxin interacting protein (TXNIP), uridine-cytidine kinase 2 (UCK2), zinc finger protein 185 (LIM domain) (ZNF185), zinc finger protein 238 (ZNF238), zinc finger protein 322B (ZNF322B) and Zwilch, kinetochore associated, homolog (ZWILCH). In some embodiments, markers are detected in a multiplex or panel format comprising 5 or more, 10 or more, 25 or more, 50 or more or all of the aforementioned markers.

In other embodiments, the present invention provides methods for determining survival of a subject diagnosed with lung cancer, comprising: contacting a sample from a subject diagnosed with lung with reagents for detection of altered expression of one or more (e.g., 2 or more, 3 or more, 4 or more, 5 or more, 10 or more, 25 or more or 50 or more or all of) of AKAP12, CYP24A1, DUSP6, ERBB3, GAPDH, H2AFZ, IL11RA, MEF2C, OGT, RRM2, SLC2A1, ABAT, ACHE, ACSM3, ADRB2, ALCAM, ARNTL2, AURKB, BCAM, BIRC5, BUB1, BZRAP1, Clorf116, CCNB1, CDKN3, CLIC2, CPS1, CTSL2, CYFIP2, DEPDC1, DRAM1, DUSP4, ECT2, EIF4A3, EPHX1, ESR1, ETV5, FAM114A2, FAM125B, FCGRT, FEN1, FMO2, GINS1, GJB3, GLS, GNG7, GPC4, GPD1L, GPR116, HK2, HMGA1, HOPX, HOXD1, HSD17B6, IL1R2, IL6R, IPCEF1, KAL1, LMF1, LYPD3, MAPK81P3, MKI67, NCAPG2, NCAPH, NLRP1, NUP37, OMD, PARVA, PCNA, PGAP3, PKP2, RAB36, RAN, RPS6KA3, RUFY3, SCNN1B, SLC34A2, SLC47A1, ST3GAL4, STC2, TCP1, TFF1, TRAM1, TRIM2, TSC22D3, TXNIP, UCK2, ZNF185, ZNF238, ZNF322B and ZWILCH. In some embodiments, altered expression of theone or more genes (e.g., relative to the expression in a sample from a subject not diagnosed with lung cancer) identifies the subject as having a decreased likelihood of survival.

Further embodiments provide the use of any of the aforementioned compositions and kits in determining the survival of a subject diagnosed with lung cancer.

Additional embodiments of the present disclosure are provided in the description and examples below.

### DESCRIPTION OF THE FIGURES

FIG. 1 shows an overview of the strategy of development and validation of 91-gene qRT-PCR classifier for lung cancer prognosis.

FIG. 2 shows major biological process of 91 survival related genes.

FIG. 3 shows survival prediction of 91-gene classifier in qRT-PCR validation set. Kaplan-Meier survival curve using patient mortality index from RSF prediction model built from training set including 91 genes, stage and age could significantly classify all 101 patients to high and low risk groups (⅓rd in each group) (A) and also 59 stage 1 patient (⅓rd in each group) (B).

FIG. 4 shows an image of qRT-PCR results for 18s-RNA control gene for all samples used in this study.

FIG. 5 shows B) repeatability of qRT-PCR for the same RNA sample C023 using all 91 survival related genes (r=0.98) and B) repeatability of qRT-PCR for the correlation of genes within the same sample but between different portion of the tumor for sample C023 using all 91 survival related genes (r=0.98).

FIG. 6 shows prediction results on two test sets by Kaplan-Meier survival curve using RSF (mortality risk index separated patient to Low, Med, High-risk groups, ⅓rd in each group) built from training set using 368 genes with stage and age. A) HR=1.00, 1.16, 1.87 and log-rank test P-value=0.12, low vs high P=0.05; B) HR=1.00, 2.25, 3.20 and log-rank test P-value=0.005, low vs high P=0.002.

FIG. 7 shows a scatter plot of the correlation between microarray value and qRT-PCR values for one gene with 47 samples (r=0.95).

FIG. 8 shows a ROC curve of 91-gene classifier on qRT-PCR validation set (2 year survival, censored patients dropped) for all patients (A) and stage 1 patients (B).

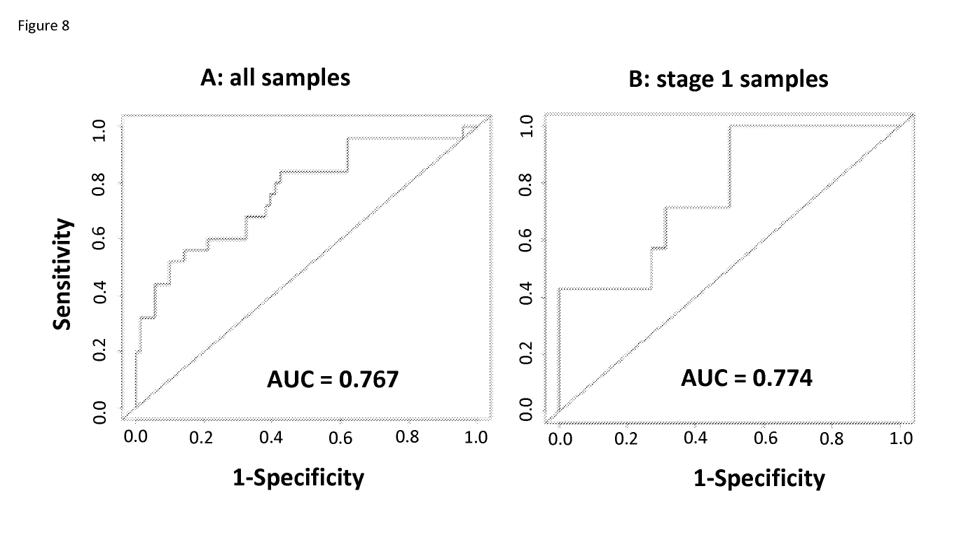

### DEFINITIONS

Unless defined otherwise, all terms of art, notations and other scientific terms or terminology used herein have the same meaning as is commonly understood by one of ordinary skill in the art to which this disclosure belongs. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

As used herein, “a” or “an” means “at least one” or “one or more.”

As used herein, the term “gene upregulated in cancer” refers to a gene that is expressed (e.g., mRNA or protein expression) at a higher level in cancer (e.g., lung cancer) relative to the level in other tissue. In this context, “other tissue” may refer to, for example, tissues from different organs in the same subject or to normal tissues of the same or different type. In some embodiments, genes upregulated in cancer are expressed at a level between at least 10% to 300% higher than the level of expression in other tissue. For example, genes upregulated in cancer are frequently expressed at a level preferably at least 25%, at least 50%, at least 100%, at least 200%, or at least 300% higher than the level of expression in other tissue.

As used herein, the term “gene upregulated in lung tissue” or “gene downregulated in lung cancer” refers to a gene that is expressed (e.g., mRNA or protein expression) at a higher or lower level in tissue obtained from lung (e.g., lung cancer tissue or cell) relative to the level in other tissue (e.g., non-cancerous lung tissue or non-lung tissue). In some embodiments, genes upregulated in lung tissue are expressed at a level between at least 10% to 300%. For example, genes upregulated in cancer are frequently expressed at a level preferably at least 25%, at least 50%, at least 100%, at least 200%, or at least 300% higher than the level of expression in other tissues. In some embodiments, genes upregulated in lung tissue are exclusively expressed in lung tissue.

As used herein, the terms “detect”, “detecting” or “detection” may describe either the general act of discovering or discerning or the specific observation of a detectably labeled composition.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “probe” refers to an oligonucleotide, whether occurring naturally as in a purified restriction digest or produced synthetically, recombinantly or by PCR amplification, which is capable of hybridizing to at least a portion of another oligonucleotide of interest. A probe may be single-stranded or double-stranded. Probes are useful in the detection, identification and isolation of particular gene sequences. It is contemplated that any probe used in methods of the present disclosure will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the methods or reagents of the present disclosure be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. An isolated nucleic acid is present in a form or setting that is different from that in which it is found in nature. In contrast, non-isolated nucleic acids are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the nucleic acid, oligonucleotide or polynucleotide often will contain, at a minimum, the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues (e.g., lung tissue biopsy), and gases. Biological samples include blood products, such as plasma, serum and the like. Such examples are not however to be construed as limiting the sample types applicable to the present invention.

### DETAILED DESCRIPTION OF THE INVENTION

The present disclosure relates to compositions and methods for cancer diagnosis, research and therapy, including but not limited to, cancer markers. In particular, the present disclosure relates to cancer markers as diagnostic markers and clinical targets for lung cancer.

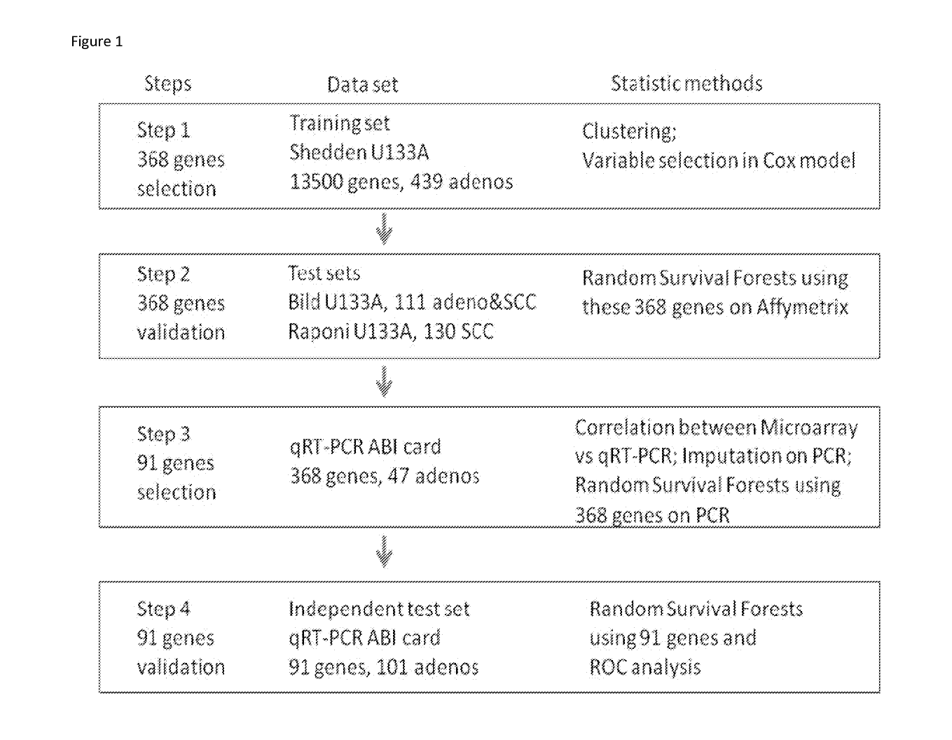

Lung cancer is a heterogeneous disease, and it is often difficult to accurately predict patient survival using tumor pathological characteristics or staging information only. Experiments conducted during the course of development of embodiments of the present invention generated a qRT-PCR card-based 91-gene survival classifier, using the four major procedures shown in FIG. 1, for the purpose of developing a clinically practicable assay for lung cancer prognosis.

Of the 91 genes in this study, the functional analysis showed that more than 20 different biological processions were involved. Most of these processes were cancer-related and most of these genes have been reported by others as individually being involved in cancer development or used for cancer diagnosis or prognosis (Table 6). The 91-gene list was compared with six other qRT-PCR based studies and one meta-analysis based on microarray (Table 7).

The gene cluster analysis and risk index created from Cox models have been often utilized as statistical approaches for gene expression profile-based survival prediction (Shedden et al., Nat Med 14:822-7, 2008). Genes in the same cluster which are similarly expressed in a dataset often represent similar biological functions or define similar pathological features. The panels described herein utilize genes representative of as many clusters as possible to aid in prediction regard of tumor heterogeneity. Both Cox models and RSF were used to aid in the identification of genes and development of the classifier. In general, performances of RSF and Cox model were similar, with RSF being complementary to the Cox model providing genes important for survival prediction based on the VIMP value (Ishwaran et al., Annals of Applied Statistics 2:841-860, 2008; Pang et al., Bioinformatics 26:250-8, 2010). The panels described herein combine clustering, Cox model and RSF prediction models for survival-related gene selection to predict survival in a qRT-PCR platform data in lung cancer.

Experiments conducted during the course of developments of embodiments of the present invention were prospectively planned and executed as described in FIG. 1. The strategy of incrementally refining and reducing the number of genes as the study transitioned from an Affymetrix platform to a qRT-PCR platform was predefined and executed as planned. The selection of individual markers was based on a large sample size used in the training set and the data was from one uniform study but measured at four centers. This decreases any microarray platform effect. Survival data from all 439 subjects in the training data rather than just the subset of those who had both Affymetrix and qRT-PCR measurements was utilzed. Further, the successful survival prediction for the 91-gene qRT-PCR platform also included stage 1 cancer. These markers find use in prediction of patient survival with SCC indicating that some similar biological processes are shared by SCC38.

Accordingly, in some embodiments, the present invention provides cancer markers and panels of cancer markers for the research, screening and clinical (e.g., prediction of patient survival with early stage lung cancer) applications.

### I. Cancer Markers

In some embodiments, the present invention provides cancer markers whose altered expression (e.g., relative to the level of expression in a non-cancerous lung sample) is indicative of cancer (e.g., lung cancer). For example, in some embodiments, the cancer marker comprises one or more (e.g., 2 or more, 3 or more, 4 or more, 5 or more, 10 or more, 25 or more or 50 or more or all of) of AKAP12, CYP24A1, DUSP6, ERBB3, GAPDH, H2AFZ, IL11RA, MEF2C, OGT, RRM2, SLC2A1, ABAT, ACHE, ACSM3, ADRB2, ALCAM, ARNTL2, AURKB, BCAM, BIRC5, BUB1, BZRAP1, Clorf116, CCNB1, CDKN3, CLIC2, CPS1, CTSL2, CYFIP2, DEPDC1, DRAM1, DUSP4, ECT2, EIF4A3, EPHX1, ESR1, ETV5, FAM114A2, FAM125B, FCGRT, FEN1, FMO2, GINS1, GJB3, GLS, GNG7, GPC4, GPD1L, GPR116, HK2, HMGA1, HOPX, HOXD1, HSD17B6, IL1R2, IL6R, IPCEF1, KAL1, LMF1, LYPD3, MAPK81P3, MKI67, NCAPG2, NCAPH, NLRP1, NUP37, OMD, PARVA, PCNA, PGAP3, PKP2, RAB36, RAN, RPS6KA3, RUFY3, SCNN1B, SLC34A2, SLC47A1, ST3GAL4, STC2, TCP1, TFF1, TRAM1, TRIM2, TSC22D3, TXNIP, UCK2, ZNF185, ZNF238, ZNF322B and ZWILCH. Table 6 describes the complete names of the aforementioned genes. Sequences of the genes can be found, for example, in the GenBank database (NCBI). In some embodiments, expression of the marker is increased or decreased relative to the level in a non-cancerous lung sample (e.g., 5%, 10%, 25%, 50%, 75%, 100% or more altered expression).

In some embodiments, genes for inclusion in the panel are selected based on their ability to predict survival in lung cancer patients. In some embodiments, statistical techniques (e.g., those described in the experimental section below) are utilized to screen the predictive value of genes or panels of genes. In some embodiments, panels are screened for their collective predictive value using any number of statistical techniques (e.g., those described herein).

In some embodiments, markers are detected in a multiplex or panel format comprising 5 or more, 10 or more, 25 or more, 50 or more or all of the aforementioned markers.

### II. Antibodies

The cancer marker proteins of the present disclosure, including fragments, derivatives and analogs thereof, may be used as immunogens to produce antibodies having use in the diagnostic, screening, research, and therapeutic methods described hereain. The antibodies may be polyclonal or monoclonal, chimeric, humanized, single chain, Fv or Fab fragments. Various procedures known to those of ordinary skill in the art may be used for the production and labeling of such antibodies and fragments. See, e.g., Burns, ed., Immunochemical Protocols, 3^{rd} ed., Humana Press (2005); Harlow and Lane, Antibodies: A Laboratory Manual, Cold Spring Harbor Laboratory (1988); Kozbor et al., Immunology Today 4: 72 (1983); Köhler and Milstein, Nature 256: 495 (1975). Antibodies or fragments exploiting the differences between the truncated or chimeric protein resulting from a cancer marker and their respective native proteins are particularly preferred.

### III. Diagnostic and Screening Applications

Expression levels of the cancer may be detectable as DNA, RNA or protein. The present disclosure provides RNA and protein based diagnostic and screening methods that detect the expression levels of the cancer markers described herein. The present disclosure also provides compositions and kits for diagnostic and screening purposes.

A. Sample

Any sample suspected of containing the cancer markers may be tested according to the methods of the present disclosure. By way of non-limiting example, the sample may be tissue (e.g., a lung biopsy sample), blood, cell secretions or a fraction thereof (e.g., plasma, serum, exosomes, etc.).

The patient sample typically involves preliminary processing designed to isolate or enrich the sample for the cancer marker(s) or cells that contain the cancer marker(s). A variety of techniques known to those of ordinary skill in the art may be used for this purpose, including but not limited to: centrifugation; immunocapture; cell lysis; and, nucleic acid target capture.

B. Detection of RNA

In some preferred embodiments, detection of lung cancer markers (e.g., including but not limited to, those disclosed herein) is detected by measuring the expression of corresponding mRNA in a tissue sample (e.g., lung tissue). mRNA expression may be measured by any suitable method, including but not limited to, those disclosed below.

In some embodiments, RNA is detection by Northern blot analysis. Northern blot analysis involves the separation of RNA and hybridization of a complementary labeled probe. An exemplary method for Northern blot analysis is provided in Example 3.

In still further embodiments, RNA (or corresponding cDNA) is detected by hybridization to a oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference).

The INVADER assay detects specific nucleic acid (e.g., RNA) sequences by using structure-specific enzymes to cleave a complex formed by the hybridization of overlapping oligonucleotide probes.

In some embodiments, microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays are utilized for measuring cancer marker mRNA levels. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink jet printing; or, electrochemistry on microelectrode arrays.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

Cancer marker nucleic acids can be detected by any conventional means. For example, the cancer markers can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174; Nelson et al., Nonisotopic Probing, Blotting, and Sequencing, ch. 17 (Larry J. Kricka ed., 2d ed. 1995, each of which is herein incorporated by reference in its entirety).

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed, for example, in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in meothd of embodiments of the present disclsoure. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products methods of embodiments of the present disclosure. See, e.g., U.S. Pat. No. 5,814,447 (herein incorporated by reference in its entirety).

In some embodiments, nucleic acid sequencing is utilized in the detection of nucleic acids.

Illustrative non-limiting examples of nucleic acid sequencing techniques include, but are not limited to, chain terminator (Sanger) sequencing and dye terminator sequencing, or high throughput sequencing methods. The present disclosure is not intended to be limited to any particular methods of sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

C. Detection of Protein

In other embodiments, gene expression of cancer markers is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein. The generation of antibodies is described above.

Illustrative non-limiting examples of immunoassays include, but are not limited to: immunoprecipitation; Western blot; ELISA; immunohistochemistry; immunocytochemistry; immunochromatography; flow cytometry; and, immuno-PCR. Polyclonal or monoclonal antibodies detectably labeled using various techniques known to those of ordinary skill in the art (e.g., colorimetric, fluorescent, chemiluminescent or radioactive labels) are suitable for use in the immunoassays.

Immunoprecipitation is the technique of precipitating an antigen out of solution using an antibody specific to that antigen. The process can be used to identify proteins or protein complexes present in cell extracts by targeting a specific protein or a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and optionally sorting microscopic particles or cells suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

In other embodiments, the immunoassay described in U.S. Pat. Nos. 5,599,677 and 5,672,480; each of which is herein incorporated by reference.

D. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a sputum sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of long term survival) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

E. Kits

In yet other embodiments, the present invention provides kits for the detection and characterization of lung cancer. In some embodiments, the kits contain antibodies specific for a cancer marker, in addition to detection reagents and buffers. In other embodiments, the kits contain reagents specific for the detection of mRNA, cDNA or protein (e.g., oligonucleotide probes, primers, antibodies, optionally in an arrary format). In preferred embodiments, the kits contain all of the components necessary, sufficient or useful to perform a detection assay, including all controls, directions for performing assays, and any necessary software for analysis and presentation of results.

### IV. Drug Screening Applications

In some embodiments, the present disclosure provides drug screening assays (e.g., to screen for anticancer drugs). The screening methods of the present disclosure utilize cancer markers described herein alone or in combination with other markers. For example, in some embodiments, the present disclosure provides methods of screening for compounds that alter (e.g., increase or decrease) the expression of cancer markers. The compounds or agents may interfere with transcription, by interacting, for example, with the promoter region. The compounds or agents may interfere with mRNA. The compounds or agents may interfere with pathways that are upstream or downstream of the biological activity of the cancer marker. In some embodiments, candidate compounds are antisense or interfering RNA agents (e.g., oligonucleotides) directed against cancer markers. In other embodiments, candidate compounds are antibodies or small molecules that specifically bind to a cancer marker regulator or expression products of the present disclosure and inhibit its biological function.

In one screening method, candidate compounds are evaluated for their ability to alter cancer marker expression by contacting a compound with a cell or subject expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method.

In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present disclosure provides screening methods for identifying modulators, i.e., candidate or test compounds or agents (e.g., proteins, peptides, peptidomimetics, peptoids, small molecules or other drugs) which bind to cancer markers of the present disclosure, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly lung cancer.

In one embodiment, the disclosure provides assays for screening candidate or test compounds that are substrates of a cancer marker protein or polypeptide or a biologically active portion thereof. In another embodiment, the disclosure provides assays for screening candidate or test compounds that bind to or modulate the activity of a cancer marker protein or polypeptide or a biologically active portion thereof.

The test compounds of the present disclosure can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

### EXPERIMENTAL

The following examples are provided in order to demonstrate and further illustrate certain preferred embodiments and aspects of the present disclosure and are not to be construed as limiting the scope thereof.

### Example 1

### Methods

Published Microarray Data Collection

Three published Affymetrix microarray data sets representing 680 tumors were used in the survival-related gene selection procedure. The primary training data set included 439 lung adenocarcinomas (Shedden et al., Nat Med 14:822-7, 2008), and a combined 111 lung adenocarcinomas and squamous carcinoma (SCC) data set represented test set one8 and a 130 lung SCC data set was used as test set two (Raponi M, Zhang Y, Yu J, et al: Gene expression signatures for predicting prognosis of squamous cell and adenocarcinomas of the lung. Cancer Res 66:7466-72, 2006). The clinical information for these three data sets is provided in Table 1. The primary outcome was overall survival for all datasets, censored at 5 years. The information of adjuvant chemotherapy or radiation therapy was not provided in the original paper.

Patients and Tissue Specimens for qRT-PCR Measurements

A subset of 47 of the 439 patients had qRT-PCR measured. These patients either died within 3 years (n=24) or survived more than 5 years (n=23). In addition an independent validation set of 101 lung adenocarcinomas procured from patients having pulmonary resection for cancer between February 1992 and November 2007 at the University of Michigan was used. This study was approved by the Institutional Review Board of University of Michigan. None of the patients received preoperative chemotherapy or radiation therapy. The information of adjuvant chemotherapy or radiation was not collected in this study. The clinical information for this cohort is also presented in Table 1.

RNA Isolation and cDNA Synthesis

RNA was extracted using miRNeasy Mini Kit (Qiagen, Cat. no. 217004, Valencia, Calif.). Frozen dissected tumor tissue was placed in 700 μl of QIAzol lysis reagent (Qiagen) and disrupted with a Teflon-glass homogenizer to facilitate dissolution. An on-column DNA digestion with the RNase-Free DNase (Qiagen, cat. no. 79254) was performed. The RNA yield and OD260/280 quality was analyzed by NanoDrop 3300 Fluorospectrometer (Thermo Scientific, Wilmington, Del.). For cDNA synthesis, 2 μg of total RNA was converted to cDNA in a 20 μl volume using the random-primed high-capacity cDNA Reverse Transcription Kit with RNase inhibitor (Applied Biosystems Ins, (ABI), PN 4374966, Foster City, Calif.).

Custom TaqMan Low Density Arrays and Quantitative RT-PCR

Custom TaqMan Low Density Arrays (384-well micro fluidic cards) were obtained from ABI (PN 4342265 Format 384 was used for 384 genes set qRT-PCR, and PN 4342259 Format 96a was used for 96 genes set qRT-PCR). The primers of survival-related genes including an endogenous loading control gene (18s RNA) and blank controls pre-coated on the cards. The preparation and running of the micro fluidic cards (qRT-PCR) followed the guidelines of product protocols (Applied Biosystems 7900HT Micro Fluidic Card Getting Started Guide, PN 4319399). Briefly, each 100-μl PCR mix 3 for each fill reservoir of the card contained 5 μl cDNA (100 ng of total RNA converted to cDNA), 50 μl TaqMan Universal PCR Master Mix (2×) (ABI, PN 4304437) and 45 μl RNase/DNase-free water. After loading a sample-specific 100 μl PCR mix to each reservoir, the card was centrifuged at 1200 rpm twice and then sealed. The sample containing fluidic cards were then run on the ABI Prism 7900HT Sequence Detection System using a two-temperature cycling protocol: 95° C. for 10 min, then 40 cycles of 97° C. for 30 sec and 60° C. for 1 min. Cycle threshold (Ct) values were generated for each card by automatic selection of a threshold.

Statistical Analysis

Initial Microarray Data Processing and Filtering

The preprocessing and filtering steps were identical to those described in Shedden et al (Nat Med 14:822-7, 2008). Microarray probe sets from the 439 tumor training set (dChip processed) were obtained and those showing fewer than five samples with greater than a 50 raw expression units were removed. The missing expression measurements were imputed using nearest neighbor averaging. All values were log-2 transformed. Standard deviations were calculated for each probe set and 25% of the probe sets with the smallest standard deviations were removed. In order to select genes consistently measured across the four centers (U of Michigan, Moffitt, Memorial Sloan Kettering and Dana Farber), the integrative correlation coefficients were calculated (R-package: Choi et al: BMC Bioinformatics 8:364, 2007). Probe sets whose consistency scores, the average of the integrative correlations over the 6 possible pair-wise comparisons across centers, less than 0.25, were removed. After pre-screening 13,306 probes were left for further analysis. All genes in training and testing datasets are median-centered and MAD-scaled for use in subsequent analyses.

Selection of Survival-related Clusters and Genes

In order to select an initial subset of genes prognostic for survival for patients with lung cancer in Affymetrix platform, two statistical methods, clustering of genes and variable selection were used. It was contemplated that: (a) highly-correlated gene expression can be separated into clusters with potentially similar biological functional groups; and, (b) clusters and subsets of genes in each selected cluster are prognostic for survival.

The selection procedure was carried out as follows:

Clustering of genes: Genes were separated into K groups using K-means clustering. The number of clusters, K, was chosen to be 300. K=300 was picked since the correlations between the 300 clusters' average gene expression values are reasonably small. A two-stage selection procedure was used; first, selection of clusters and second, selection of genes within each of the selected clusters. Backward elimination and stepwise regression on 300 averaged expressions in the Cox proportional hazard model were implemented for cluster selection. Stage and age variables were included in the Cox model, but no selection was made on them. Within each of selected clusters, the second selection identified a subset of genes prognostic for survival based on various criteria: (1) its correlation to the center of cluster was greater than 0.5; (2) genes with Affymetrix probes were preferred; (3) its median expression and standard deviation across centers were similar (4) more genes were selected from bigger sized clusters (about 15-20%); (5) genes with smaller p-values (mostly less than 0.05) in Cox model adjusted for stage and age, within cluster. All 5 conditions were considered simultaneously. This approach led to a set of clusters and subsets of genes for each selected clusters considered relevant to patient survival of lung cancer.

Normalization and Imputation of qRT-PCR Values

Affymetrix measurements on 368 genes were obtained for 439 lung cancer patients, and of these, 47 patients were selected to have complete qRT-PCR measurements for all the 368 genes. The 47 patients were selected to include 24 who died early and 23 who lived more than 5 years. The qRT-PCR measurements on the remaining 392 patients were then treated as missing data. In order to have complete PCR measurements for all the patients, a multiple imputation procedures were performed for the remaining 392 patients who did not have qRT-PCR measured.

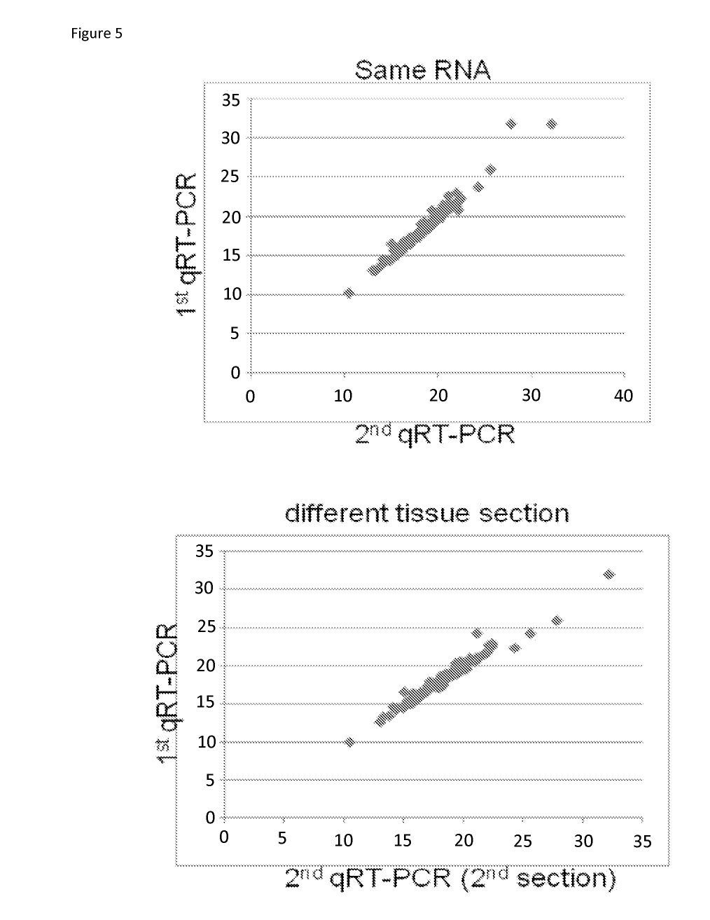

Before imputation, a method of normalization for the PCR data that resulted in the best correlation between PCR and Affymetrix measurements was developed. First, the mean PCR level for 18S, a ubiquitous gene, was subtracted from each measurement. Next, the mean expression level was calculated for each gene across all 47 samples. These overall means are then subtracted from the individual 18S normalized measurements leaving one with the residuals for each gene and patient. Finally, the average residual for each gene was calculated and subtracted from each individual's 18S normalized PCR expression level for that gene. Through this method it was possible to achieve the highest levels of Spearman's correlation between PCR and Affymetrix measurements (Table 2 and Supplementary FIG. 5).

The imputation was performed using IVEware, which uses a sequential regression imputation method. The multiple imputation algorithm was run on the normalized PCR data. The imputation approach incorporated both Affymetrix and PCR measurements as well as stage, age, and survival time. Ten iterations of the sequential regression scheme were run to create each imputed dataset and a total of 20 imputed sets were created.

Random Survival Forests (RSF) for Survival Analysis and Prediction

The random survival forests (RSF) method developed by Ishwaran H et al. (Annals of Applied Statistics 2:841-860, 2008) in R package was used to relate the expression data to survival and to give a model for prediction. The RSF is an ensemble tree method for analysis of right-censored survival data. Each of the 1000 decision trees of the forest was grown by splitting patients by comparing survival differences via log-rank test based on a randomly selected subset of variables at each node. Three different RSF's were built, one based on 439 patients and 368 genes using Affymetrix data, one based on 439 patients and 368 genes using imputed qRT-PCR data, one based on 439 patients and 91 genes using imputed qRT-PCR data. All RSF's also included age and stage as additional variables.

Once the RSF prediction model was built, test sets were dropped down to the trees for prediction. The cumulative hazard function (CHF) was derived from each tree of the RSF, and an ensemble CHF, an average over 1000 survival trees was determined. Mortality (mortality risk index in this study, or MRI) was obtained as a weighted sum over ensemble CHF, weighted by the number of individuals at risk at the different time points. Higher mortality values implied higher risk.

To test the significance of the mortality risk index it was used as a continuous covariate in a Cox model. For graphical representation the mortality risk index was used to separate patients into three tertiles (high, med, and low risk).

Each tree provides a measure of its predictive error as described by Ishwaran (supra), with smaller number indicating a better tree. The prediction error is calculated by 1-C-index (the Harrell's concordance index) in out-of-bag data which were not used for building a tree each time.

Variable importance scores (VIMPs) for all the variables used to grow trees were also generated. Large VIMPs indicate variables are good predictors for outcome whereas zero or negative values identify non-predictability. These scores were used to select genes relevant to survival in the final gene selection step.

In order to set a cut-off on VIMP for gene selection in final step, a set of “noisy” variables from uniform distribution was created and added to each of 20 imputed datasets. The VIMPs for those “noisy” variables were expected to be very low. Genes whose VIMPs were larger then averaged 20 VIMPs for “noisy” variables were selected.

The number 91 for the gene selection size was chosen because it is a practical number to measure with the typical size (two 18s RNA, two blank controls and one test primer included in the card) of a qRT-PCR card-based TaqMan Low Density Array (384-8 well micro fluidic cards) platform. With this platform one can either run four individual samples or run two samples in duplicate on each card.

Calculation of Area Under the Curve (AUC)

To evaluate the discriminative ability of the predictions from the RSF's, a receiver operating characteristic (ROC) curve was constructed for the validation dataset of 101 subjects. For this comparison the vital status (dead or alive) of the person was considered at two years; 5 subjects were removed who were censored before 2 years. The ROC curves were constructed by varying the cut-off of the MRI, and the AUC calculated.

Results

Repeatability and Performance of qRT-PCR Card-based Platform

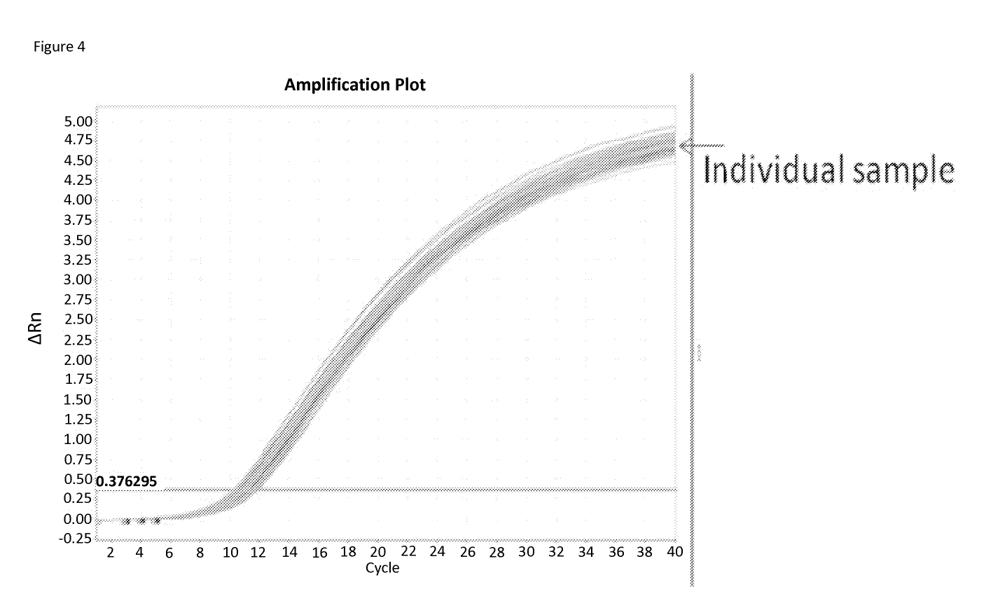

The technical performance and the repeatability of the qRT-PCR card-based for 10 tumor samples and 10 normal samples were tested using different qRT-PCR cards that were examined with the same cDNA sample and also examined different tissue sections of the same tumor. Excellent correlation and reliable values were found (FIG. 4, 5).

Pre-selection of a Subset of 368 Survival-related Genes

To minimize potential microarray batch affects, only one uniformly measured data set including 439 lung adenocarcinomas was used as the training set (Shedden et al., Nat Med 14:822-7, 2008). By K-means clustering, 300 clusters were generated based on the training data set. A total of 73 clusters, whose average gene expression was found to be related to patient survival, were then selected. From these clusters, a total of 368 genes were selected by various criteria described in the methods.

In order to test survival predictability of these 368 genes, two independent Affymetrix platform-based datasets (Bild et al., 110 samples (Bild et al., Nature 439:353-7, 2006) and Raponi et al., 130 samples (Raponi et al. Cancer Res 66:7466-72, 2006)] were tested using RSF. The RSF prediction model was built on a training data set, and both test sets were tested from the built RSF prediction model. The prediction error rates were 41.1% and 34.6%, respectively for the Bild and Raponi data sets. For both test sets, low, intermediate and high-risk groups were clearly separated by MRI with Kaplan-Meier survival curves (for Bild's data, HR=1.00, 1.16, 1.87 and log-rank test P-value=0.12, low vs high P=0.05; for Raponi's data, HR=1.00, 2.25, 3.20 and log-rank test P-value

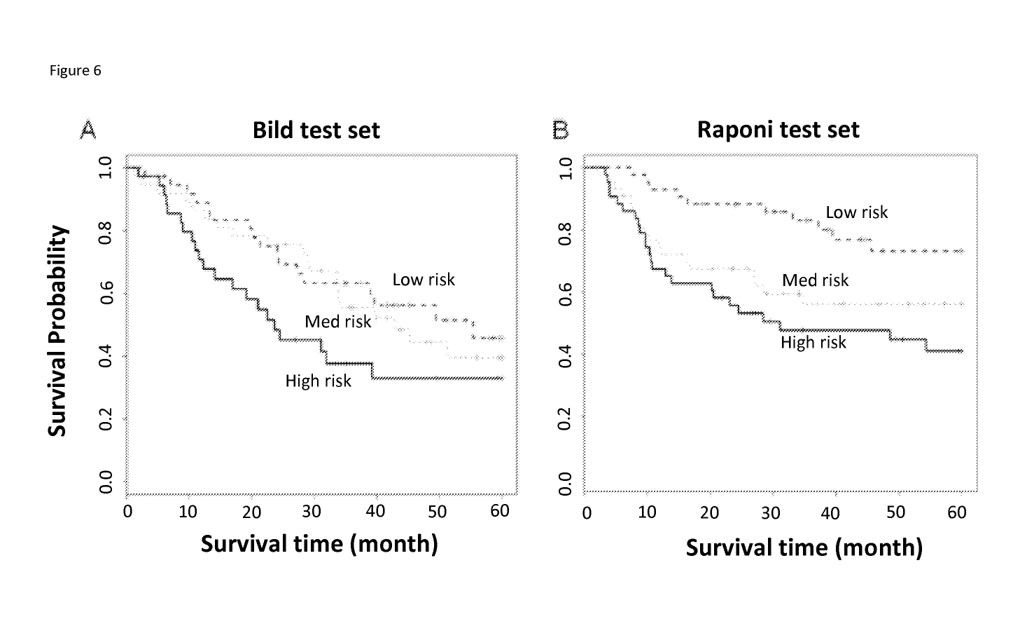

=0.005, low vs high P=0.002) (Table 4 and FIG. 6). This demonstrated the suitability of these 368 genes as predictors for patient survival of lung cancer.

The prediction results using the Raponi test set, although all squamous cell lung cancers, was better than using the Bild data set which included both adenocarcinomas and squamous cell lung cancers.

Identification of a Subset of 91 Survival-related Genes

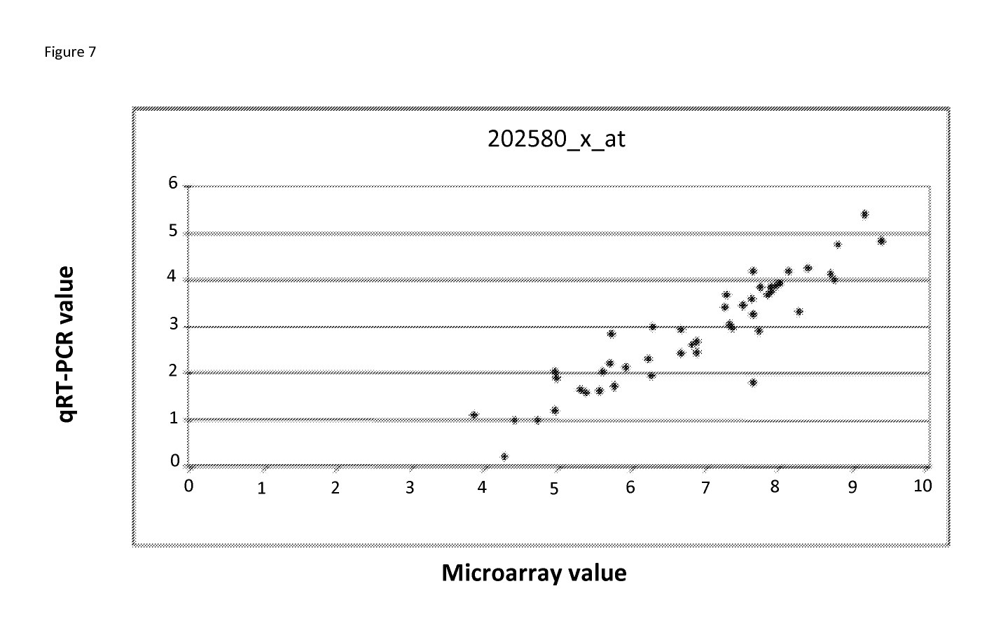

In order to identify a 91-gene qRT-PCR platform-based classifier from a subset of the 368 genes selected in the Affymetrix platform, three major processes were performed. First, genes whose qRT-PCR measurement showed high correlation with Affymetrix microarray measurements based on 47 samples from the training set were defined. There were 301 out of 368 (301/368, 82%) genes which had a significantly high correlation with correlation value larger than 0.5 (P<0.001, Table 2 and FIG. 7). This indicated that the hybridization efficiency of some probes in a microarray can be different.

Second, based on these measured qRT-PCR expression values, the qRT-PCR values were inputted for the remaining patients in the training dataset. A RSF was performed using 1000 trees and it was repeated 10 times on each of the 20 imputed training data sets. Genes were selected based on four criteria: (a) correlations between qRT-PCR and Affymetrix measurements were greater than 0.5, (b) P values from Cox model adjusted for stage and age on the imputed PCR data were less than 0.05, (c) average variable importance measure (VIMP) from the RSF (mean of 10 VIMPs per dataset) larger than the “noise” VIMP average from RSF, and (d) the number of genes selected from each cluster was roughly proportional to the cluster size with a representative from each cluster if possible. A set of 91 genes from 53 clusters were identified.

Finally, in order to compare the power of prediction of this 91-gene classifier to 368-gene classifier based on the microarray data, a RSF prediction analysis as done with 368-gene signature was performed. The 91-gene signature gave a similar prediction results as the 368 genes did on the two test sets. The prediction error rates were 40.7% and 36.3%, respectively for Bild and Raponi test sets (Table 5). This indicated that the 91-gene signature was comparable to the 368-gene signature in predicting patient survival in lung cancer.

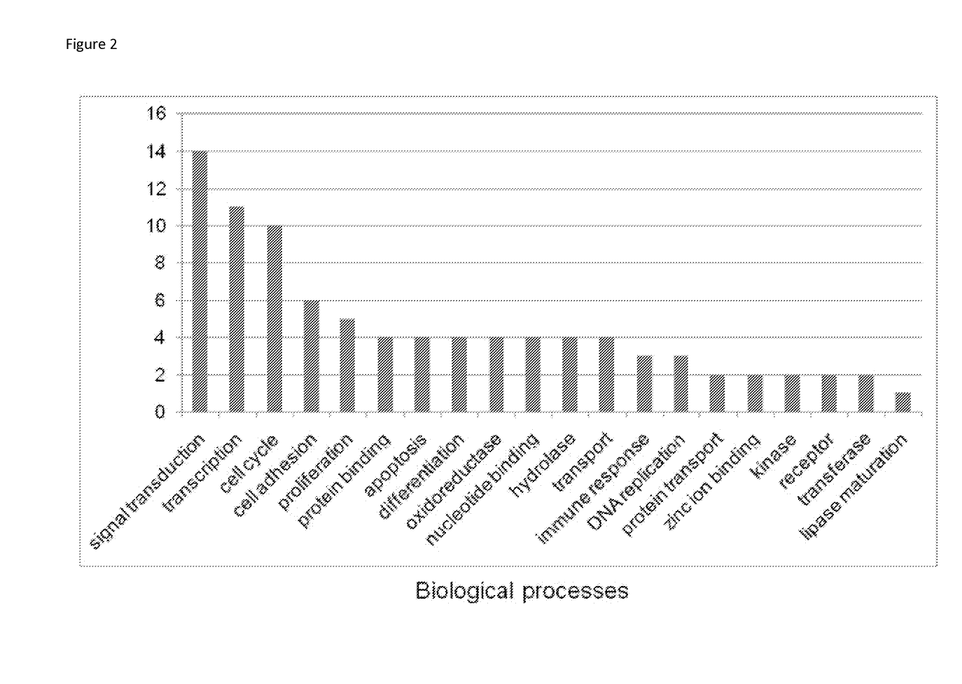

The annotation of these 91 genes is shown in Table 6, and the main biological categories are indicated in FIG. 2. Among these, signal transduction, transcription regulation, cell cycle, cell adhesion, and proliferation are the major biological processes.

Development and Validation of the qRT-PCR Platform-based Classifier in an Independent Test Set

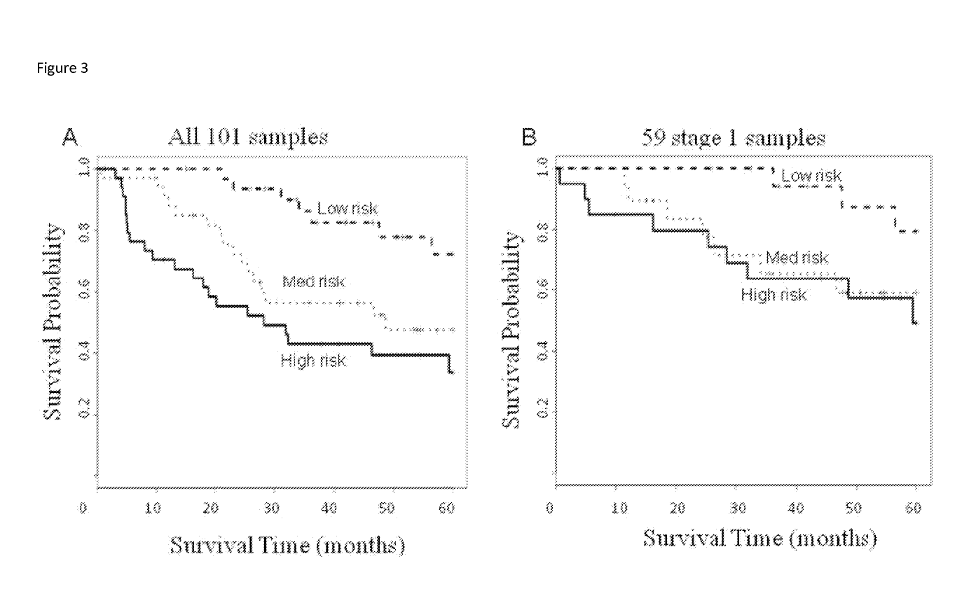

In order to validate the 91-gene classifier for lung cancer prognosis, the qRT-PCR card-based platform was utilized with an independent cohort of 101 lung adenocarcinomas. The qRT-PCR data was normalized as described above. The RSF with the 91 genes, stage and age information were built on the average of 20 imputed training sets of 439 tumors. The data obtained from the new qRT-PCR card-based 101 tumor cohort was then dropped down the RSF model for prediction. The prediction error rate for the 101 qRT-PCR test cohort was 26.6%. The utility of RSF predictors was tested using a univariate Cox model with the MRI as a continuous measure. The RSF prediction was significant for the 101 patient's cohort (likelihood ratio test (LRT) P<0.0001). Using the MRI produced from RSF, three risk groups were also identified, with patient 5-year survival being significantly different between low, med, and high-risk groups (HR=1.00, 2.82, 4.42; FIG. 3A and Table 3). For stage I tumors only, this MRI was also significantly related to survival (Cox model LRT, P=0.001) and separated patients into low, med, and high risk groups (HR=1.00, 3.29, 3.776, FIG. 3B and Table 3). The area under the curve (AUC)s from receiver operating characteristic (ROC) analyses were both 0.77 for all patients and for stage 1 only (FIG. 7). A notable feature of the validation shown in FIG. 3 is the large separation between the curves in the first two years of follow-up, with almost no patients dying in the first two years for the low-risk group, but with significant number of deaths in the first two years for the high-risk group.

In order to evaluate whether the set of 91 genes improves the prediction compared to clinical variables, age and stage in the validation dataset, the two Cox models were compared via LRT; a model with age and stage versus a model with age, stage and the mortality index from RSF based on 91 genes only. It was found that the set of 91 genes improves the prediction compared to age and stage only (LRT P<0.0001) on all 101 patients.

TABLE 1
Clinical Characteristics of Samples Used this Study

| | Training | Bild test | Raponi test | Validation |
|---|---|---|---|---|
| Data set | set | set | set | set |
| Platform | U133A | U133 | U133A | qRT-PCR |
| | | plus2.0 | | |
| Sample number | 439 | 111 | 130 | 101 |
| | | 58 Ad/53 | | |
| Type of cancer | Ad | SCC | SCC | Ad |
| Age average (SD) | 64.4 (10.1) | 64.8 (9.6) | 67.5 (9.9) | 67.0 (9.6) |
| Gender | | | | |
| Female | 218 (49.7%) | 48 (43.2%) | 48(36.9%) | 53(52.5%) |
| Male | 221 | 63 | 82 | 48 |
| Stage | | | | |
| Stage I | 276(62.9%) | 67(63.2%) | 73(56.2%) | 59(58.4%) |
| Stage II | 104 | 18 | 34 | 16 |
| Stage III | 59 | 21 | 23 | 26 |
| Differentiaton | | | | |
| Well | 60 | NA | 15 | 28 |
| Moderate | 208 | NA | 76 | 38 |
| Poor | 166(38.3%) | NA | 39(30%) | 34(33.7%) |
| Dead (5 year) | 186(42.4%) | 58(52.3%) | 52(40%) | 44(43.6%) |
| Alive | 253 | 53 | 78 | 57 |
| Median survival (m) | 47 | 31.1 | 34.5 | 28.8 |

Abbreviation:
Ad, adenocarcinomas;
SCC, squamous cell cancer.

TABLE 2
Spearman Correlation
Between qRT-PCR and Microarray

| Spearman | Number of |
|---|---|
| correlation | genes |
| >0.9 | 67 |
| 0.8-0.9 | 92 |
| 0.7-0.8 | 71 |
| 0.6-0.7 | 46 |
| 0.5-0.6 | 25 |
| 0.4-0.5 | 23 |
| 0.3-0.4 | 15 |
| 0.2-0.3 | 13 |
| <0.2 | 16 |

TABLE 3
Prediction Results of 91-gene Signature for
qRT-PCR Valdation Set to (n = 101)

| | RSF* | Cox model** | log-rank test*** | | |
|---|---|---|---|---|---|
| | Test error rate | P | HR | 95% Cl | P |
| | 26.60% | <0.0001 | | | |
| Low-risk | | | 1 | | 0.001 |
| Med-risk | | | 2.82 | 1.16-6.88 | |
| High-risk | | | 4.42 | 1.88-10.42 | |

*RSF prediction model built from 439 training set including 91 genes, stage and age;
**Mortality risk index (MRI) as continous value, likelihood ratio test (LRT) was used;
***MRI separated test patients to 3 risk groups (Low, Med and High-risk ⅓rd in each group)

TABLE 4

| | Bild test set (n = 111) | | | | | Raponi test set (n = 130) | | | | |
|---|---|---|---|---|---|---|---|---|---|---|
| | RSF* | | log-rank | | | RSF* | | log-rank | | |
| | Test error | Cox | test*** | | | Test error | Cox | test*** | | |
| | rate | model** P | HR | 95% CI | P | rate | model** P | HR | 95% CI | P |
| Low risk | 41.10% | 0.11 | 1 | | 0.12 | 34.60% | 0.0002 | 1 | | 0.005 |
| Med risk | | | 1.16 | 0.61-2.21 | | | | 2.25 | 1.04-4.58 | |
| High risk | | | 1.87 | 0.98-3.55 | | | | 3.2 | 1.53-6.71 | |

MRI = mortality risk index
*RSF prediction model built from 439 training set including 368 genes, stage and age using Affimetrix microarray data
**MRI as continuous value, likelihood ratio test (LRT) was used
***MRI separated test patients to 3 risk groups (Low, Med and High-risk, ⅓rd in each group)

TABLE 5

| | Bild test set (n = 111) | | | | | Raponi test set (n = 130) | | | | |
|---|---|---|---|---|---|---|---|---|---|---|
| | RSF* | | log-rank | | | RSF* | | log-rank | | |
| | Test error | Cox | test*** | | | Test error | Cox | test*** | | |
| | rate | model** P | HR | 95% CI | P | rate | model** P | HR | 95% CI | P |
| Low risk | 40.70% | 0.14 | 1 | | 0.14 | 36.30% | 0.009 | 1 | | 0.006 |
| Med risk | | | 1.78 | 0.93-3.39 | | | | 2.63 | 1.23-5.62 | |
| High risk | | | 1.64 | 0.93-3.62 | | | | 2.92 | 1.38-6.17 | |

MRI = mortality risk index
*RSF prediction model built from 439 training set including 91 genes, stage and age using Affimetrix microarray data
**MRI as continuous value, likelihood ratio test (LRT) was used
***MRI separated test patients to 3 risk groups (Low, Med and High-risk, ⅓rd in each group)

TABLE 6

| | | | | | | Cancer |
|---|---|---|---|---|---|---|
| Gene | | | | | | related |
| Symbol | Probe Set ID | Cluster | VIMP | Gene Title | Major function | reported |
| NLRP1 | 210113_s_at | 138 | 0.0016 | NLR family, pyrin domain containing 1 | apoptosis | leukaemia |
| BIRC5 | 202094_at | 182 | 0.0007 | baculoviral IAP repeat-containing 5 | apoptosis | cancers |
| | | | | (survivin) | | |
| DRAM | 213627_at | 164 | 0.0005 | DNA-damage regulated autophagy | apoptosis | cancers |
| | | | | modulator 1 | | |
| CYFIP2 | 215785_s_at | 161 | −0.0008 | cytoplasmic FMR1 interacting protein 2 | apoptosis | cancers |
| PKP2 | 207717_s_at | 23 | 0.0001 | plakophilin 2 | cell adhesion | cancers |
| ALCAM | 201951_at | 187 | 0.0001 | activated leukocyte cell adhesion | cell adhesion | cancers |
| | | | | molecule | | |
| PARVA | 217890_s_at | 11 | −0.0003 | parvin, alpha | cell adhesion | breast cancer |
| KAL1 | 205206_at | 141 | −0.0006 | Kallmann syndrome 1 sequence | cell adhesion | |
| OMD | 205907_s_at | 67 | −0.0006 | osteomodulin | cell adhesion | |
| BCAM | 203009_at | 98 | −0.0006 | basal cell adhesion molecule (Lutheran | cell adhesion | cancers |
| | | | | blood group) | | |
| CDKN3 | 209714_s_at | 121 | 0.0027 | cyclin-dependent kinase inhibitor 3 | cell cycle | cancers |
| NCAPG2 | 219568_s_at | 122 | 0.0025 | non-SMC condensin II complex, subunit | cell cycle | melanoma |
| | | | | G2 | | |
| NCAPH | 212949_at | 182 | 0.0004 | non-SMC condensin I complex, subunit H | cell cycle | melanoma |
| BUB1 | 209642_at | 121 | −0.0002 | budding uninhibited by benzimidazoles 1 | cell cycle | cancers |
| | | | | homolog (yeast) | | |
| AURKB | 209464_at | 121 | −0.0004 | aurora kinase B | cell cycle | cancers |
| ZWILCH | 218349_s_at | 244 | −0.0005 | Zwilch, kinetochore associated, homolog | cell cycle | breast cancer |
| | | | | (Drosophila) | | |
| NUP37 | 218622_at | 230 | −0.0007 | nucleoporin 37 kDa | cell cycle | |
| CCNB1 | 214710_s_at | 182 | −0.0008 | cyclin B1 | cell cycle | cancers |
| RAN | 200750_s_at | 97 | −0.0011 | RAN, member RAS oncogene family | cell cycle | cancers |
| GAPDH | 213453_x_at | NA | −0.0025 | glyceraldehyde-3-phosphate | cell cycle | cancers |
| | | | | dehydrogenase | | |
| H2AFZ | 200853_at | 122 | 0.0003 | H2A histone family, member Z | differentiation | breast cancer |
| DUSP6 | 208893_s_at | 199 | 0.0003 | dual specificity phosphatase 6 | differentiation | cancers |
| LYPD3 | 204952_at | 23 | 0.0003 | LY6/PLAUR domain containing 3 | differentiation | cancers |
| RUFY3 | 213939_s_at | 166 | 0.0000 | RUN and FYVE domain containing 3 | differentiation | nervous |
| | | | | | | system |
| | | | | | | development |
| GINS1 | 206102_at | 182 | 0.0010 | GINS complex subunit 1 (Psf1 homolog) | DNA replication | melanoma |
| FEN1 | 204768_s_at | 281 | 0.0000 | flap structure-specific endonuclease 1 | DNA replication | cancers |
| RRM2 | 209773_s_at | 182 | −0.0003 | ribonucleotide reductase M2 | DNA replication | cancers |
| PERLD1 | 55616_at | 106 | 0.0020 | post-GPI attachment to proteins 3 | hydrolase | gastric cancer |
| DUSP4 | 204014_at | 127 | 0.0011 | dual specificity phosphatase 4 | hydrolase | lung cancer |
| EPHX1 | 202017_at | 98 | 0.0009 | epoxide hydrolase 1, microsomal | hydrolase | cancers |
| | | | | (xenobiotic) | | |
| GLS | 203159_at | 119 | 0.0002 | glutaminase | hydrolase | cancers |
| FCGRT | 218831_s_at | 189 | 0.0010 | Fc fragment of IgG, receptor, transporter, | immune response | |
| | | | | alpha | | |
| IL6R | 205945_at | 164 | −0.0002 | interleukin 6 receptor | immune | cancers |
| | | | | | response | |
| IL1R2 | 205403_at | 162 | −0.0003 | interleukin 1 receptor, type II | immune response | |
| UCK2 | 209825_s_at | 147 | −0.0001 | uridine-cytidine kinase 2 | kinase | |
| HK2 | 202934_at | 278 | −0.0011 | hexokinase 2 | kinase | cancers |
| LMF1 | 46142_at | 84 | −0.0009 | lipase maturation factor 1 | lipase maturation | |
| EIF4A3 | 201303_at | 97 | 0.0012 | eukaryotic translation initiation factor 4A, | nucleotide | GI cancer |
| | | | | isoform 3 | binding | |
| ACSM3 | 205942_s_at | 33 | 0.0003 | acyl-CoA synthetase medium-chain family | nucleotide binding | |
| | | | | member 3 | | |
| FAM114A2 | 213588_s_at | 79 | 0.0002 | family with sequence similarity 114, | nucleotide binding | |
| | | | | member A2 | | |
| CPS1 | 204920_at | 127 | −0.0006 | carbamoyl-phosphate synthetase 1, | nucleotide | GI cancer |
| | | | | mitochondrial | binding | |
| CYP24A1 | 206504_at | 261 | 0.0017 | cytochrome P450, family 24, subfamily A, | oxidoreductase | cancers |
| | | | | polypeptide 1 | | |
| GPD1L | 212510_at | 98 | 0.0013 | glycerol-3-phosphate dehydrogenase 1- | oxidoreductase | |
| | | | | like | | |
| FMO2 | 211726_s_at | 219 | 0.0003 | flavin containing monooxygenase 2 (non- | oxidoreductase | oral cancer |
| | | | | functional) | | |
| HSD17B6 | 205700_at | 141 | 0.0001 | hydroxysteroid (17-beta) dehydrogenase | oxidoreductase | |
| | | | | 6 homolog (mouse) | | |
| TFF1 | 205009_at | 215 | 0.0003 | trefoil factor 1 | proliferation | cancers |
| PCNA | 201202_at | 244 | 0.0000 | proliferating cell nuclear antigen | proliferation | cancers |
| MKI67 | 212022_s_at | 281 | −0.0004 | antigen identified by monoclonal antibody | proliferation | cancers |
| | | | | Ki-67 | | |
| ACHE | 205377_s_at | 159 | −0.0004 | acetylcholinesterase (Yt blood group) | proliferation | cancers |
| GPC4 | 204984_at | 141 | −0.0005 | glypican 4 | proliferation | |
| CTSL2 | 210074_at | 140 | 0.0275 | cathepsin L2 | protein binding | cancers |
| GJB3 | 205490_x_at | 200 | 0.0014 | gap junction protein, beta 3, 31 kDa | protein binding | thyroid |
| | | | | | | cancer |
| TCP1 | 208778_s_at | 140 | −0.0014 | t-complex 1 | protein binding | cancers |
| SLC2A1 | 201250_s_at | 278 | −0.0023 | solute carrier family 2 (facilitated glucose | protein binding | cancers |
| | | | | transporter), member 1 | | |
| FAM125B | 221667_s_at | 26 | 0.0015 | family with sequence similarity 125, | protein transport | |
| | | | | member B | | |
| TRAM1 | 201398_s_at | 192 | −0.0008 | translocation associated membrane | protein transport | ovarian |
| | | | | protein 1 | | |
| C1orf116 | 219476_at | 153 | 0.0000 | chromosome 1 open reading frame 116 | receptor | |
| BZRAP1 | 205839_s_at | 229 | −0.0012 | benzodiazapine receptor (peripheral) | receptor | |
| | | | | associated protein 1 | | |
| DEPDC1 | 220295_x_at | 281 | 0.0065 | DEP domain containing 1 | signal | bladder |
| | | | | | transduction | cancer |
| OGT | 209240_at | 192 | 0.0008 | O-linked N-acetylglucosamine (GlcNAc) | signal transduction | |
| | | | | transferase | | |
| RPS6KA3 | 203843_at | 202 | 0.0007 | ribosomal protein 56 kinase, 90 kDa, | signal | Coffin-Lowry |
| | | | | polypeptide 3 | transduction | syndrome |
| IL11RA | 204773_at | 161 | 0.0006 | interleukin 11 receptor, alpha | signal | cancers |
| | | | | | transduction | |
| CLIC2 | 213415_at | 146 | 0.0004 | chloride intracellular channel 2 | signal transduction | |
| GPR116 | 212950_at | 153 | 0.0000 | G protein-coupled receptor 116 | signal transduction | |
| MAPK8IP3 | 213178_s_at | 3 | 0.0000 | mitogen-activated protein kinase 8 | signal transduction | |
| | | | | interacting protein 3 | | |
| AKAP12 | 210517_s_at | 56 | −0.0003 | A kinase (PRKA) anchor protein 12 | signal | cancers |
| | | | | | transduction | |
| RAB36 | 211471_s_at | 229 | −0.0004 | RAB36, member RAS oncogene family | signal | rhabdoid |
| | | | | | transduction | tumors |
| ERBB3 | 202454_s_at | 194 | −0.0007 | v-erb-b2 erythroblastic leukemia viral | signal | cancers |
| | | | | oncogene homolog 3 (avian) | transduction | |
| GNG7 | 206896_s_at | 138 | −0.0008 | guanine nucleotide binding protein (G | signal | oesophageal |
| | | | | protein), gamma 7 | transduction | cancer |
| STC2 | 203439_s_at | 278 | −0.0009 | stanniocalcin 2 | signal | cancers |
| | | | | | transduction | |
| ADRB2 | 206170_at | 53 | −0.0012 | adrenergic, beta-2-, receptor, surface | signal | cancers |
| | | | | | transduction | |
| ECT2 | 219787_s_at | 244 | −0.0031 | epithelial cell transforming sequence 2 | signal | cancers |
| | | | | oncogene | transduction | |
| ZNF322A | 219376_at | 258 | 0.0017 | zinc finger protein 322B | transcription | |
| ARNTL2 | 220658_s_at | 117 | 0.0007 | aryl hydrocarbon receptor nuclear | transcription | entrainment |
| | | | | translocator-like 2 | | of circadian |
| | | | | | | clock |
| ESR1 | 205225_at | 199 | 0.0003 | estrogen receptor 1 | transcription | cancers |
| ZNF238 | 212774_at | 228 | 0.0002 | zinc finger protein 238 | transcription | brain tumor |
| TXNIP | 201010_s_at | 189 | 0.0001 | thioredoxin interacting protein | transcription | cancers |
| TSC22D3 | 203763_s_at | 72 | −0.0001 | TSC22 domain family, member 3 | transcription | ovarian |
| | | | | | | cancer |
| ETV5 | 203348_s_at | 82 | −0.0001 | ets variant 5 | transcription | cancers |
| HOPX | 211597_s_at | 223 | −0.0002 | HOP homeobox | transcription | cancers |
| MEF2C | 209200_at | 138 | −0.0002 | myocyte enhancer factor 2C | transcription | hepatocellular |
| | | | | | | carcinoma |
| HMGA1 | 206074_s_at | 281 | −0.0004 | high mobility group AT-hook 1 | transcription | therapeutic |
| | | | | | | target in |
| | | | | | | pancreatic |
| | | | | | | cancer |
| HOXD1 | 205975_s_at | 98 | −0.0005 | homeobox D1 | transcription | differentiation |
| | | | | | | and limb |
| | | | | | | development |
| ST3GAL4 | 203759_at | 200 | 0.0002 | ST3 beta-galactoside alpha-2,3- | transferase | |
| | | | | sialyltransferase 4 | | |
| ABAT | 209460_at | 299 | −0.0013 | 4-aminobutyrate aminotransferase | transferase | |
| PIP3-E | 214735_at | 199 | −0.0002 | interaction protein for cytohesin exchange | transport | |
| | | | | factors 1 | | |
| SLC34A2 | 204124_at | 119 | −0.0005 | solute carrier family 34 (sodium | transport | ovarian |
| | | | | phosphate), member 2 | | cancer |
| SLC47A1 | 219525_at | 286 | −0.0010 | solute carrier family 47, member 1 | transport | |
| SCNN1B | 205464_at | 223 | −0.0010 | sodium channel, nonvoltage-gated 1, beta | transport | renal cell |
| | | | | | | carcinoma |
| ZNF185 | 203565_at | 9 | 0.0010 | zinc finger protein 185 (LIM domain) | zinc ion binding | cancers |
| TRIM2 | 215945_s_at | 202 | 0.0001 | tripartite motif-containing 2 | zinc ion binding | |

Note:
The number of cluster was came from K-means clustering based on 439 training set.
The values of VIMP were obtained from RSF prediction model on the 101 qRT-PCR validation set.
Cancer related reported list was obtained from PubMed searches that included tumorigenesis, diagnosis or prognosis related reports for that gene.

TABLE 7

| | | | | | | | | | | | meta- |
|---|---|---|---|---|---|---|---|---|---|---|---|
| | | | | qRT-PCR | qRT-PCR | qRT-PCR | qRT-PCR | qRT-PCR | qRT-PCR | qRT-PCR | microarray |
| | | | | 91 | 8 survival | 5 survival | 3 survival | 4 survival | 6 survival | 6 recurrence | 64 survival |
| | | | | survival | genes | genes | genes | genes | genes | genes | genes |
| | | microarray | | genes | Endoh | chen HY | Lau | Raz | Boutros | Lee | Lu Y |
| Platform | Gene | Shedden data | | this | H | NEJM | SK | D | PC PNAS | ES | PloSMed |
| Probe Set ID | Symbol | p value* | beta | study | JCO 2004 | 2007 | JCO 2007 | CCR 2008 | 2009 | CCR 2008 | 2006 |
| 208891_at | DUSP6 | 6.00E−04 | −0.26 | 1 | | 1 | | | | | |
| 202454_s_at | ERBB3 | 0.0017 | −0.29 | 1 | | 1 | | 1 | | | |
| 212801_at | CIT | 0.00029 | −0.21 | | 1 | | | | | | |
| 218498_s_at | ERO1L | 0.0013 | 0.34 | | 1 | | | | | | |
| 207338_s_at | PTGES | 0.028 | 0.18 | | 1 | | | | | | |
| 207011_s_at | PTK7 | 8.00E−04 | −0.37 | | 1 | | | | | | |
| 203453_at | SCNN1A | 0.075 | −0.11 | | 1 | | | | | | |
| 204026_s_at | ZWINT | 0.0023 | 0.24 | | 1 | | | | | | |
| 204890_s_at | LCK | 0.72 | 0.04 | | | 1 | | 1 | | | |
| 203414_at | MMD | 0.11 | 0.14 | | | 1 | | | | | |
| 200887_s_at | STAT1 | 0.5 | 0.08 | | | 1 | | | | | |
| 206337_at | CCR7 | 0.076 | −0.15 | | | | 1 | | | | |
| 200989_at | HIF1A | 0.21 | 0.22 | | | | 1 | | 1 | | |
| 204729_s_at | STX1A | 0.01 | 0.40 | | | | 1 | | 1 | | |
| 212724_at | RND3 | 0.01 | 0.20 | | | | | 1 | | | |
| 200910_at | CCT3 | 0.67 | −0.07 | | | | | | 1 | | |
| 201137_s_at | HLA-DPB1 | 0.11 | −0.12 | | | | | | 1 | | |
| 206750_at | MAFK | 0.82 | −0.03 | | | | | | 1 | | |
| 209111_at | RNF5 | 0.26 | −0.16 | | | | | | 1 | | |
| 205626_s_at | CALB1 | 0.73 | −0.02 | | | | | | | 1 | |
| 210072_at | CCL19 | 0.02 | −0.19 | | | | | | | 1 | |
| 203924_at | GSTA1 | 0.46 | −0.09 | | | | | | | 1 | |
| 214453_s_at | IFI44 | 0.56 | −0.04 | | | | | | | 1 | |
| 204259_at | MMP7 | 0.29 | 0.05 | | | | | | | 1 | |
| 207355_at | SLC1A7 | 0.072 | −0.16 | | | | | | | 1 | |
| 201250_s_at | SLC2A1 | 4.10E−07 | 0.37 | 1 | | | | | | | 1 |
| 209200_at | MEF2C | 5.60E−05 | −0.38 | 1 | | | | | | | 1 |

*sex, age and stage were included in multivariable Cox model, n = 439 adenocarcinomas, 5 year survival
‘1’ indicated overlapping genes used in original paper

Although a variety of embodiments have been described in connection with the present disclosure, it should be understood that the claimed invention should not be unduly limited to such specific embodiments. Indeed, various modifications and variations of the described compositions and methods of the invention will be apparent to those of ordinary skill in the art and are intended to be within the scope of the following claims.

We claim:

1. A kit for determining survival of a subject diagnosed with lung cancer, comprising: reagents for detection of altered expression of one or more genes selected from the group consisting of ABAT, ACHE, ACSM3, ADRB2, ALCAM, ARNTL2, AURKB, BCAM, BIRC5, BUB1, BZRAP1, Clorf116, CCNB1, CDKN3, CLIC2, CPS1, CTSL2, CYFIP2, DEPDC1, DRAM1, DUSP4, ECT2, EIF4A3, EPHX1, ESR1, ETV5, FAM114A2, FAM125B, FCGRT, FEN1, FMO2, GINS1, GJB3, GLS, GNG7, GPC4, GPD1L, GPR116, HK2, HMGA1, HOPX, HOXD1, HSD17B6, IL1R2, IL6R, IPCEF1, KAL1, LMF1, LYPD3, MAPK81P3, MKI67, NCAPG2, NCAPH, NLRP1, NUP37, OMD, PARVA, PCNA, PGAP3, PKP2, RAB36, RAN, RPS6KA3, RUFY3, SCNN1B, SLC34A2, SLC47A1, ST3GAL4, STC2, TCP1, TFF1, TRAM1, TRIM2, TSC22D3, TXNIP, UCK2, ZNF185, ZNF238, ZNF322B and ZWILCH.

2. The kit of claim 1, wherein said kit comprises reagents for detection of 5 or more of said genes.

3. The kit of claim 1, wherein said kit comprises reagents for detection of 10 or more of said genes.

4. The kit of claim 1, wherein said kit comprises reagents for detection of 25 or more of said genes.

5. The kit of claim 1, wherein said kit comprises reagents for detection of 50 or more of said genes.

6. The kit of claim 1, wherein said reagents are affixed to a solid support.

7. The kit of claim 6, wherein said solid support is an array.

8. A method for determining survival of a subject diagnosed with lung cancer, comprising: contacting a sample from a subject diagnosed with lung with reagents for detection of altered expression of one or more genes selected from the group consisting of ABAT, ACHE, ACSM3, ADRB2, ALCAM, ARNTL2, AURKB, BCAM, BIRC5, BUB1, BZRAP1, Clorf116, CCNB1, CDKN3, CLIC2, CPS1, CTSL2, CYFIP2, DEPDC1, DRAM1, DUSP4, ECT2, EIF4A3, EPHX1, ESR1, ETV5, FAM114A2, FAM125B, FCGRT, FEN1, FMO2, GINS1, GJB3, GLS, GNG7, GPC4, GPD1L, GPR116, HK2, HMGA1, HOPX, HOXD1, HSD17B6, IL1R2, IL6R, IPCEF1, KAL1, LMF1, LYPD3, MAPK81P3, MKI67, NCAPG2, NCAPH, NLRP1, NUP37, OMD, PARVA, PCNA, PGAP3, PKP2, RAB36, RAN, RPS6KA3, RUFY3, SCNN1B, SLC34A2, SLC47A1, ST3GAL4, STC2, TCP1, TFF1, TRAM1, TRIM2, TSC22D3, TXNIP, UCK2, ZNF185, ZNF238, ZNF322B and ZWILCH.

9. The method of claim 8, wherein altered expression of said one or more genes identifies said subject as having a decreased likelihood of survival.

10. A kit for determining survival of a subject diagnosed with lung cancer, comprising: reagents for detection of altered expression of AKAP12, CYP24A1, DUSP6, ERBB3, GAPDH, H2AFZ, IL11RA, MEF2C, OGT, RRM2, SLC2A1, ABAT, ACHE, ACSM3, ADRB2, ALCAM, ARNTL2, AURKB, BCAM, BIRC5, BUB1, BZRAP1, Clorf116, CCNB1, CDKN3, CLIC2, CPS1, CTSL2, CYFIP2, DEPDC1, DRAM1, DUSP4, ECT2, EIF4A3, EPHX1, ESR1, ETV5, FAM114A2, FAM125B, FCGRT, FEN1, FMO2, GINS1, GJB3, GLS, GNG7, GPC4, GPD1L, GPR116, HK2, HMGA1, HOPX, HOXD1, HSD17B6, IL1R2, IL6R, IPCEF1, KAL1, LMF1, LYPD3, MAPK81P3, MKI67, NCAPG2, NCAPH, NLRP1, NUP37, OMD, PARVA, PCNA, PGAP3, PKP2, RAB36, RAN, RPS6KA3, RUFY3, SCNN1B, SLC34A2, SLC47A1, ST3GAL4, STC2, TCP1, TFF1, TRAM1, TRIM2, TSC22D3, TXNIP, UCK2, ZNF185, ZNF238, ZNF322B and ZWILCH.

11. The kit of claim 10, wherein said reagents are affixed to a solid support.

12. The kit of claim 11, wherein said solid support is an array.

1-12. (canceled)

13. A kit for determining survival of a subject diagnosed with lung cancer, comprising: reagents for detection of altered expression of ABAT, ACHE, ACSM3, ADRB2, AKAP12, ALCAM, ARNTL2, AURKB, BCAM, BIRC5, BUB1, BZRAP1, Clorf116, CCNB1, CDKN3, CLIC2, CPS1, CTSL2, CYFIP2, CYP24A1, DEPDC1, DRAM1, DUSP4, DUSP6, ECT2, EIF4A3, EPHX1, ERBB3, ESR1, ETV5, FAM114A2, FAM125B, FCGRT, FEN1, FMO2, GAPDH, GINS1, GJB3, GLS, GNG7, GPC4, GPD1L, GPR116, H2AFZ, HK2, HMGA1, HOPX, HOXD1, HSD17B6, IL1R2, IL11RA, IL6R, IPCEF1, KAL1, LMF1, LYPD3, MAPK81P3, MEF2C, MKI67, NCAPG2, NCAPH, NLRP1, NUP37, OGT, OMD, PARVA, PCNA, PERLD1, PIP3E, PGAP3, PKP2, RAB36, RAN, RPS6KA3, RRM2, RUFY3, SCNN1B, SLC2A1, SLC34A2, SLC47A1, ST3GAL4, STC2, TCP1, TFF1, TRAM1, TRIM2, TSC22D3, TXNIP, UCK2, ZNF185, ZNF238, ZNF322B and ZWILCH.

15. The kit of claim 13, wherein said reagents are affixed to a solid support.

16. The kit of claim 16, wherein said solid support is an array.

17. The kit of claim 13, wherein said reagents are selected from the group consisting of nucleic acid probes, a pair of amplification primers, and one or more sequencing primers.

18. The kit of claim 17, wherein said reagent is labeled.

19. A method for determining survival of a subject diagnosed with lung cancer, comprising:
a) contacting a sample from a subject diagnosed with lung with reagents for detection of altered expression of ABAT, ACHE, ACSM3, ADRB2, AKAP12, ALCAM, ARNTL2, AURKB, BCAM, BIRC5, BUB1, BZRAP1, Clorf116, CCNB1, CDKN3, CLIC2, CPS1, CTSL2, CYFIP2, CYP24A1, DEPDC1, DRAM1, DUSP4, DUSP6, ECT2, EIF4A3, EPHX1, ERBB3, ESR1, ETV5, FAM114A2, FAM125B, FCGRT, FEN1, FMO2, GAPDH, GINS1, GJB3, GLS, GNG7, GPC4, GPD1L, GPR116, H2AFZ, HK2, HMGA1, HOPX, HOXD1, HSD17B6, IL1R2, IL11RA, IL6R, IPCEF1, KAL1, LMF1, LYPD3, MAPK81P3, MEF2C, MKI67, NCAPG2, NCAPH, NLRP1, NUP37, OGT, OMD, PARVA, PCNA, PERLD1, PIP3E, PGAP3, PKP2, RAB36, RAN, RPS6KA3, RRM2, RUFY3, SCNN1B, SLC2A1, SLC34A2, SLC47A1, ST3GAL4, STC2, TCP1, TFF1, TRAM1, TRIM2, TSC22D3, TXNIP, UCK2, ZNF185, ZNF238, ZNF322B and ZWILCH; and
b) determining survival of said subject when said altered expression is detected.

20. The method of claim 19, wherein altered expression of said genes identifies said subject as having a decreased likelihood of survival.

21. The method of claim 19, wherein said reagents are selected from the group consisting of nucleic acid probes, a pair of amplification primers, and one or more sequencing primers.

22. The method of claim 19, wherein said altered expression is increased expression or decreased expression.
